(19) United States
(12) Plant Patent Application Publication
(10) Pub. No.: US 2002/0174472 P1
(43) Pub. Date: Nov. 21, 2002

(54) Bracteantha plant named 'Antique Shades'

(75) Inventor: Matthew Turner, New South Wales (AU)

Correspondence Address: ROCKEY, MILNAMOW & KATZ, LTD., Two Prudential Plaza, 180 North Stetson, Suite 4700, Chicago, IL, 60601, US

(73) Assignee: Oasis Horticulture PTY, Ltd.

(21) Appl. No.: 09/819,272

(22) Filed: Mar. 28, 2001

(51) Int. Cl. A01H 5/00

(52) U.S. Cl. PLT/359

(57) ABSTRACT

A new and distinct Bracteantha plant named Antique Shades, characterized by its pale purple flowers, upright growth habit, and medium green leaves.

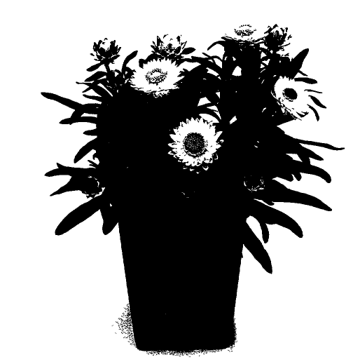

### BACKGROUND OF THE INVENTION

The present invention comprises a new and distinct Bracteantha plant, botanically known as Bracteantha bracteata and hereinafter referred to by the cultivar name Antique Shades characterized by its pale purple flowers.

The new cultivar was developed by the inventor through a controlled breeding program at New South Wales, Australia.

The female (seed) parent of Antique Shades was the cultivar Spectrum (not patented in the United States). The mate (pollen) parent of Antique Shades was the cultivar Diamond Head (not patented in the United States). Antique shades was selected as one of the flowering progeny of the above cross.

Asexual reproduction of the new cultivar by terminal cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits pale purple flowers,

(b) forms medium green foliage,

(c) exhibits a good basal branching character and

(d) exhibits an upright growth habit.

When the new cultivar of the present invention is compared to Florabella Pink, it is found that the new cultivar is more compact, exhibits different pubescense on stems and leaves and has flowers of a darker purple color as detailed in TABLE A.

### BRIEF DESCRIPTION OF PHOTOGRAPH

The accompanying photograph show as nearly true as it is reasonably possible to make the same in color illustrations of this type, typical flower and foliage characteristics of the new cultivar. The plants were grown for 12 weeks in a greenhouse at West Chicago, Ill., U.S.A.

### DETAILED BOTANICAL DESCRIPTION

The Antique Shades cultivar has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Feb. 15, 2001. The readings were taken between 10:00 and 11:00 a.m. under natural daylight conditions. The plants were produced from cuttings taken from stock plants and grown in a poly carbonate covered greenhouse under conditions comparable to those used in commercial practice while utilizing a soilless growth medium and maintaining temperatures of approximately 72° F. during the day and approximately 65° F. during the night.

Classification:

Botanical.—Bracteantha bracteata cultivar Antique Shades.

Commercial.—Helichrysum.

Parentage:

Female parent.—Spectrum.

Male Parent.—Diamond Head.

Propagation:

Type cutting.—Terminal tip.

Time to Initiate Roots.—Approximately 30 to 50 days with the shorter times generally being experienced in the summer and the longer times in the winter.

Plant description:

Habit of Growth.—Moderate. A mature plant, 14 weeks after the planting of a rooted cutting, commonly measures approximately 18 cm in height and approximately 30 cm in diameter.

Branching habit.—Freely basal branching although pinching improves basal branching. Commonly develops approximately 9 lateral branches.

Form.—Upright, mounded.

Lateral Stem.—Diameter: Approximately 4 mm with hirsute pubescence. Color: 145B. Internode length: Approximately 4 mm.

Foliage.—Arrangement: Alternate. Shape: Lanceolate with entire margin, broadly acute apex and clasping base. Size: Approximately 7.2 cm in length and approximately 9 mm at widest point. Upper surface is rough and 137A. Lower surface is smooth 137C. Venation of both surfaces: Closest to 145C.

Flowering description:

Flowering Habit.—Freely flowering.

Natural Flowering Season.—Spring through fall.

Inflorescence type.—Single, composite, persistent.

Flowers arrangement.—Acropetally on a capitulum.

Bud.—Size: 2 cm in length, 2 cm in diameter. Color: Closest to 71A.

Inflorescence diameter.—Approximately 3.7 cm.

Ray florets.—Quantity: Approximately 11 whorls. Aspect: Appear flat at maturity then become downwardly pointing with age. Appearance: Smooth, papery. Shape: Linear. Size: Length-1.1 cm, width varies from 1 to 4 mm. Color-Upper surface, fully opened ray floret: between 12C and 121D with tips of ; lower surface:

Capitulum diameter.—1.2 cm.

Disc florets.—Quantity: Numerous. Shape: Tubular. Length: Approximately 6 mm.

Peduncle.—Length: Approximately 6 cm. Strength: good. Aspect: Erect. Color: 144C. Texture: Puberulent.

Fragrance.—None.

Reproductive Organs.—Stigma color 25A. Pollen color: 21A.

TABLE A

| CHARACTERISTIC | ANTIQUE SHADES | FLORABELLA PINK |
|---|---|---|
| PLANT HEIGHT-CM | 18 | 23 |
| PLANT | 30 | 22 |
| DIAMETER-CM | | |
| # LATERAL | 9 | 10 |
| BRANCHES | | |
| INTERNODE | 4 mm | 1.5 cm |
| LENGTH-CM | | |
| STEM TEXTURE | HIRSUTE | TOMENTOSE |
| LEAF LENGTH | 7.2 | 8 |
| PEDUNCLE | 6 | 7.5 |
| LENGTH-CM | | |
| PEDUNCLE TEXTURE | PUBERULENT | TOMENTOSE |
| INFLORESCENCE | 3.7 | 4.2 |
| DIAMETER-CM | | |

I claim:

1. A new and distinct cultivar of Bractheantha named Antique Shades substantially as herein shown and described, which:
(a) exhibits pale purple flowers,
(b) forms medium green foliage,
(c) exhibits a good basal branching character and
(d) exhibits an upright growth habit.
